SEQUENCE LISTING (10 sequences)

SEQ ID NO: 1 - DNA, 20 bp, Artificial Sequence
  CD29 sense primer
aatgaagggcgtgttggtag

SEQ ID NO: 2 - DNA, 20 bp, Artificial Sequence
  CD29 antisense primer
cgttgctggcttcacaagta

SEQ ID NO: 3 - DNA, 20 bp, Artificial Sequence
  CD44 sense primer
gcaatgcttctcagaccaca

SEQ ID NO: 4 - DNA, 20 bp, Artificial Sequence
  CD44 antisense primer
ctggccaatgtagttcacag

SEQ ID NO: 5 - DNA, 21 bp, Artificial Sequence
  LPL sense primer
atggagagcaaagccctgctc

SEQ ID NO: 6 - DNA, 21 bp, Artificial Sequence
  LPL antisense primer
gttaggtccagctggatcgag

SEQ ID NO: 7 - DNA, 21 bp, Artificial Sequence
  CBFA1 sense primer
cagttcccaagcatttcatcc

SEQ ID NO: 8 - DNA, 20 bp, Artificial Sequence
  CBFA1 antisense primer
tcaatatggtcgccaaacag

SEQ ID NO: 9 - DNA, 21 bp, Artificial Sequence
  GAPDH sense primer
agccgcatcttcttttgcgtc

SEQ ID NO: 10 - DNA, 21 bp, Artificial Sequence
  GAPDH antisense primer
tcatatttggcaggtttttct

(19) United States
(12) Patent Application Publication
(10) Pub. No.: US 2010/0285582 A1
(43) Pub. Date: Nov. 11, 2010

(54) VARIOUS HUMAN DENTAL STEM CELLS HAVING A MINERALIZATION ABILITY AND THE METHOD FOR CULTURING THEM

(75) Inventor: Pill-Hoon Choung, Seoul (KR)

Correspondence Address: LUCAS & MERCANTI, LLP, 475 PARK AVENUE SOUTH, 15TH FLOOR, NEW YORK, NY, 10016, US

(73) Assignee: SEOUL NATIONAL UNIVERSITY INDUSTRY FOUNDATION, Seoul (KR)

(21) Appl. No.: 12/680,938

(22) PCT Filed: Oct. 1, 2007
(86) PCT No.: PCT/KR2007/004796

§ 371 Date: Jul. 6, 2010

(51) Int. Cl. C12N 5/02; C12N 5/00

(52) U.S. Cl. 435/375; 435/325; 435/378; 435/383

(57) ABSTRACT

The present invention relates to various human dental stem cells having a mineralization ability and a method for culturing the same, more precisely postnatal stem cells having a mineralization ability, which are separated from human dental tissues such as dental pulp (DPSCs), periodontal ligament (PDLSCs), periapical follicle (PAFSCs) and mandibular bone marrow (MBMSCs) and a method for culturing the same under the optimum growth conditions. The human dental stem cells of the present invention can be obtained without additional injury as well as new stem cell sources such as teeth extracted from orthodontic purposes, prophylactically extracted nondecayed third molar teeth and discarded bone segments from orthognathic surgery, so that they can be effectively used for regeneration of injured teeth.

### TECHNICAL FIELD

The present invention relates to various human dental stem cells having a mineralization ability and a method for culturing the same, more precisely postnatal stem cells having a mineralization ability, which are separated from human dental tissues such as dental pulp (DPSCs), periodontal ligament (PDLSCs), periapical follicle (PAFSCs) and mandibular bone marrow (MBMSCs) and a method for culturing the same under the optimal growth conditions.

### BACKGROUND ART

A tooth, developed through the process of the epithelial-mesenchymal interaction, consists of enamel, dentin, cementum and pulp. The tooth is supported by the periodontium including the periodontal ligament, cementum, alveolar bone and gingival. The tooth composes a functional unit along with the periodontium that is anchored in the alveolar bone of the maxilla or mandible.

Loss of a tooth, a jawbone, or both due to periodontal disease, dental caries, trauma, cancer or a variety of genetic disorders adversely affects not only mouth function but also the aesthetics of one's life. To reconstruct these defects, current treatments rely on autologous tissue grafts and/or metallic implants. These have some limitations such as insufficient biocompatibility, resorption of bone, limited graft quantity and donor-site morbidity. Stem cell-based research, however, has made steady progress toward solving these problems. The stem cell-based, bioengineered tooth is a promising technique for regenerative dentistry that will hopefully replace injured teeth and metal implants in the future. To date, many studies have demonstrated that postnatal stem cells are present in various tissues, including bone marrow, neural tissue, skin, retina, and dental epithelium and that these cells exhibit astonishing capacities for self-renewal and development into diverse tissues. Further, most of these studies have reported the effectiveness of stem cell regenerative therapy. But there are some disadvantages associated with harvesting stem cells, such as additional damage to the body and insufficient number of cells.

Adult stem cells, including hematopoietic stem cells in bone marrow, MSCs and cord blood stem cells derived from the umbilical cord, have been widely investigated. Along with the efforts to make bioengineered organs such as cartilage, bone, skeletal muscle, skin, nerve and cardiac muscle with postnatal stem cells, the regeneration of a biocompatible tooth has been studied for many years with various results.

A tooth is a complex tissue composed of soft tissue and hard tissue, two distinct but interdependent cell populations. Despite extensive knowledge of tooth development and of the various specialized tooth-associated cell types, little is known about the characteristics of their respective precursor cell populations in the postnatal dental tissue. Understanding the properties of dental stem cells is important for dental tissue regeneration and will help to produce an effective bioengineered tooth.

Mineralization is the process that a substance is converted from an organic substance to an inorganic substance by the deposition of mineral ions and formation of mineral crystals. It is necessary mechanism for making the hard tissue in the body. The tooth is a complex tissue composed of the soft tissues and the hard tissues. The hard tissues, including enamel, dentin, cementum and alveolar bone, provide the entire structure of the tooth and are all associated with the functioning tooth. These hard tissues of the tooth are formed by mineralization mechanisms such as amelogenesis, odontogenesis, cementogenesis and osteogenesis. Mineralization plays an important role in the mechanism of differentiation in both the tooth development and stem cell based tooth regeneration.

The present inventors, therefore, focused on utilizing tissues that can be obtained without additional injury, as well as new stem cell sources such as teeth extracted for orthodontic purposes, prophylactically extracted nondecayed third molar teeth and discarded bone segments from orthognathic surgery. The present inventors completed this invention by confirming that the separated postnatal dental stem cells were mesenchymal stem cells that had a mineralization ability.

### DISCLOSURE

### Technical Problem

It is an object of the present invention to provide human dental stem cells having a mineralization ability and a method for culturing the same under the optimal conditions.

### Technical Solution

To achieve the above object, the present invention provides postnatal stem cells isolated from human dental tissues such as dental pulp (DPSCs), periodontal ligament (PDLSCs), periapical follicle (PAFSCs) and mandibular bone marrow (MBMSCs).

The present inventors cultured the dental stem cells in the mineralization condition. Then, ability of these stem cells to mineralize was measured by Alizarin Red staining, quantitative analysis of Alizarin Red, alkaline phosphatase (ALP) activity and RT-PCR.

As a result, the deposits from DPSC and PAFSC were sparsely scattered over the adherent layer, whereas MBMSC and PDLSC cultures produced extensive sheets of calcified deposits throughout the adherent layer after 2 weeks of induction (See FIG. 4A-D). Quantification of the amount of Alizarin red after 21 days of induction showed that the accumulated calcium levels in cultures of MBMSC and PDLSC were higher than in other cultures (See Table 1).

Alkaline phosphatase (ALP) found on osteoblast membrane is widely used as a marker of biomineralization. ALP cleaves non-specifically phosphate ions from compounds and its activity is increased at the mineralization surface when a mineral crystal is growing. After 21 days of induction, ALP activity was high in MBMSCs and PDLSCs, which showed a high level of calcium accumulation. ALP activity of DPSCs was higher than PAFSCs although accumulated calcium level of DPSCs was lower than PAFSCs (See Table 1).

All of the dental stem cells showed upregulated Cbfa 1 expression in the mineral induction group. Cbfa 1 is a known transcription factor associated with differentiation of osteoblasts and is found in cementoblasts, ameloblasts and odontoblasts.

The present inventors also confirmed the characteristics of human dental stem cells. The isolated cells formed single-cell-derived colonies and most of the cells retained their fibroblastic spindle shape (See FIG. 1D). The isolated cell populations expressed the cell surface molecule STRO-1, an MSC marker, previously found to be present in DPSC (See FIG. 1E). They also expressed the MSC markers CD29 and CD44 (See FIGS. 1F and 1G). The stem cells were capable of forming adherent colonies (See FIG. 2A), characteristic of other stromal cell populations. Compared to other dental stem cells, PAFSCs (periapical follicle stem cells) formed twice the number of colonies for the same number of initial loading cells (see FIG. 2A). To evaluate proliferation abilities passage 3 PDLSC (periodontal ligament stem cells), DPSC (dental pulp stem cells), PAFSC (periapical follicle stem cells), and MBMSC (mandibular bone marrow stem cells) were plated at 1000 cells/well and cultured for 4, 7, and 10 days with optimal culture medium. PAFSC showed a higher proliferation rate at 10 days (See FIG. 2B) and a higher number of population doublings (See FIG. 2C) compared to the others.

The present inventors also studied the potential of dental stem cells to differentiate into adipocytes. After three weeks of culture with an adipogenic-inductive cocktail, Oil red O positive lipid clusters were identified in all dental stem cell cultures (See FIG. 4E-H). This correlated with an upregulation in the expression of the adipocyte-specific transcript, LPL, and the osteogenesis-specific transcript, CBFA, as detected by RT-PCR (See FIG. 4I). These results suggest that all of the tested dental stem cells possessed the ability to differentiate into developmentally diverse phenotypes, even though their capacity for differentiation varied.

In this invention, the inventors isolated and characterized stem cells from human dental pulp, periodontal ligament, periapical follicle and mandibular bone marrow. The connective tissue of PDL contains osteoblasts, cementocytes, osteoclasts, undifferentiated mesenchymal cells and fibroblasts. Interestingly, PDLSC makes abundant calcium nodules in the mineralized culture medium. It also has the potential to differentiate into other cell lineages such as adipocytes. This finding is correlated with an upregulation in the expression of the adipocyte-specific transcript, LDL, as detected by RT-PCR. DPSCs are also capable of differentiating into osteoblasts/odontoblasts and adipocytes. In particular, the inventors expected that PAFSC would have a higher differentiation capability because the undifferentiated cells were obtained from the developing tissue. Handa et al. (Handa et al., Progenitor cells from dental follicle are able to form cementum matrix in vivo. Connect Tissue Res 43, 406, 2002) reported that dental tissue from the root surface of bovine tooth germ was a cementum-like-matrix and contained cementoblast progenitors that were able to differentiated into cementoblasts. They also indicated that cells from the dental tissue are phenotypically distinct from the osteoblasts in alveolar bone and PDL cells. The inventors have shown that PAFSC cultures have excellent proliferation rates and capacity for development into adipogenic cells. These results suggest that PAFSC may have a broader capacity for differentiation than other stem cells. Interestingly, the growth of PAFSC requires plenty of FCS, a nutritional supplement, and much less ascorbic acid compared to other dental stem cells. This result is similar to the optimal culture conditions for MBMSC from children. This is probably due to a difference between matured tissues and developing tissues, although both of them are composed of postnatal stem cells. PAFSC, therefore, may be more effective in regenerating the tissues of dental root-cementum, dentin, and even PDL-than DPSC, which was suggested by Gronthos et al. to be useful for dentin regeneration (Gronthos, S., Mankani, M., Brahim, J., Robey, P. G., Shi, S. Postnatal human dental pulp stem cells (DPSCs) in vitro and in vivo. PNAS 97(25), 13625, 2000).

Several studies have been conducted on bone marrow stem cells obtained from long bone, but not from jaw (mandible and maxilla) bone. There are two major modes of bone formation, intramembranous ossification and endochondral ossification. Endochondral ossification involves the formation of cartilage tissue from aggregated mesenchymal cells and the subsequent replacement of cartilage tissue by bone, which is the type of bone formation that occurs in long bones such as the femur and tibia. Almost all jawbones are formed by intramembranous ossification, except for a small part of the mandible located in the condyle and center part of it. Additionally, the tissues in the oral cavity have the efficacy of rapid regeneration when damaged. The present inventors would therefore expect that maxillary bone regeneration from MBMSC is more efficient than from any other cells.

In this invention, the present inventors isolated a novel population of multipotent stem cells, PAFSC and MBMSC. The inventors compared with properties of various dental stem cells and showed that DPSC, PDLKSC, PFSC and MBMSC are similar to other MSCs with respect to their expression of STRO-1. But there are significant differences in stem cell properties depending on the tissue source. These results suggest the use of optimal dental tissue as a source of stem cells for developing bioengineered organs.

The present invention also provides a method for isolating postnatal dental stem cells from dental pulp (DPSCs), periodontal ligament (PDLSCs), periapical follicle (PAFSCs) or mandibular bone marrow (MBMSCs), comprising the following steps:

1) separating dental pulp (DPSCs), periodontal ligament (PDLSCs), periapical follicle (PAFSCs) or mandibular bone marrow (MBMSCs) from adult teeth;

2) digesting the tissues obtained in step 1); and

3) filtering the digested tissues of step 2).

In a preferred embodiment of the present invention, the inventors isolated dental pulp (DPSCs), periodontal ligament (PDLSCs), periapical follicle (PAFSCs) or mandibular bone marrow (MBMSCs) from tissues that can be obtained without additional injury as well as stem cell sources such as teeth extracted for orthodontic purposes, prophylactically extracted nondecayed third molar teeth and discarded bone segments from orthognathic surgery. These obtained tissues or segments were digested in a solution of 1-10 mg/ml collagenase type I and 1-10 mg/ml dispase at 37° C. for up to 2 hours, more preferably in a solution of 2-5 mg/ml collagenase type I and 2-5 mg/ml dispase at 37° C. for one hour. Then, single-cell suspensions of postnatal dental stem cells were obtained by passing the cells through a 70-um cell strainer (Falcon, BD Labware, Franklin lakes, N.J., USA).

The present invention further provides a method for culturing the dental stem cells in an optimal growth medium.

Particularly, the present invention provides a method for culturing the postnatal dental stem cells isolated from dental pulp (DP), periodontal ligament (PDL) and mandibular bone marrow (MBM) in alpha-MEM supplemented with 10% FCS, 50-200 uM ascorbic acid, 1-10 mM/L glutamine, 50-200 U/mL penicillin and 50-200 ug/mL streptomycin, more preferably supplemented with 10% FCS, 100-150 uM ascorbic acid, 1-5 mM/L glutamine, 80-120 U/mL penicillin and 80-120 ug/mL streptomycin, most preferably supplemented with 10% FCS, 100 uM ascorbic acid, 2 mM/L glutamine, 100 U/mL penicillin and 100 ug/mL streptomycin.

The present invention also provides a method for culturing the postnatal stem cells isolated from periapical follicle (PAF) in alpha-MEM supplemented with 20% FCS, 10-200 uM ascorbic acid, 1-10 mM/L glutamine, 50-200 U/mL penicillin and 50-200 ug/mL streptomycin, more preferably supplemented with 20% FCS, 10-50 uM ascorbic acid, 2-5 mM/L glutamine, 80-120 U/mL penicillin and 80-120 ug/mL streptomycin, most preferably supplemented with 20% FCS, 50 uM ascorbic acid, 2 mM/L glutamine, 100 U/mL penicillin and 100 ug/mL streptomycin.

To determine optimal culture conditions, the present inventors used PRMI, DMEM and alpha-MEM containing FCS (10% and 20%) and ascorbic acid of different concentrations (0, 50, 100 and 200 uM). Almost all the dental stem cells showed optimum growth when cultured in alpha-MEM supplemented with 10% FCS, 100 uM ascorbic acid, 2 mM/L glutamine, 100 U/mL penicillin and 100 ug/mL streptomycin. PAFSC, however, showed optimum growth when cultured in alpha-MEM supplemented with 20% FCS and 50 uM ascorbic acid (See FIG. 3).

### ADVANTAGEOUS EFFECT

The novel human dental stem cells that can be obtained without additional injury and be isolated from teeth extracted for orthodontic purposes, prophylactically extracted nondecayed third molar teeth and discarded bone segments from orthognathic surgery are effectively used for tooth regeneration.

### DESCRIPTION OF DRAWINGS

The application of the preferred embodiments of the present invention is best understood with reference to the accompanying drawings, wherein:

FIG. 1 is a diagram illustrating that postnatal dental stem cells were isolated from human dental tissues and identified as stem cells by immunohistochemistry and RT-PCR. Stem cells were isolated from different tissues such as PDL (A), PAF (B) and dental pulp (C). PAF (D) stem cells had a fibroblastic spindle shape similar to stem cells from other cell sources such as DPSC, PDLSC and MBMSC. Stem cells (E) isolated from mandibular bone marrow (MBMSC) expressed STRO-1, an MSC marker. Isolated stem cells also expressed the MSC markers CD29 (F), CD44 (G) and GAPDH (H). Lane 1: DPSC, Lane 2: PDLSC, Lane 3: MBMSC, Lane 4: PAFSC.

FIG. 2 is a diagram illustrating that dental stem cells were highly proliferative and produced comparable colonies in their culture conditions. (A) Clonogenic efficiency was assessed. Initially, 1×10⁴ cells were plated in 100 mm dishes and colony numbers were counted at day 10. Only colonies with more than 50 cells were included in the colony number. (B) The proliferation rates of dental stem cells at different time intervals (4, 7 and 10 days). (C) The total number of isolated dental stem cells was counted every 5 days after seeding with 3×10⁴ cells.

FIG. 3 is a diagram illustrating optimal culture conditions tested for different dental stem cells. (A) DPSC, (B) PDLSC, (C) PAFSC, (D) MBMSC. Almost all of the dental stem cells showed optimal growth when cultured in 10% FCS and 100 uM ascorbic acid in alpha-MEM.

FIG. 4 is a diagram illustrating multipotent differentiation of dental stem cells. (A-D) Alizarin red staining showed mineral nodule formation with induction. (E-H) After 3 weeks in the induction medium, lipid accumulation was noted by Oil red O staining. (I) Expression of the adipocyte specific transcript, LPL, and the osteogenesis-specific transcript, CBFA, was detected by RT-PCR. C: noninduction control, I: induction.

FIG. 5 is a diagram illustrating proliferation and colony forming capacity of various dental stem cells. A) Total cell number of isolated dental stem cells was counted every 5 days after seeding with 3×10⁴ cells. Growth rates of dental stem cells declined as passage increased. B) Colony population of isolated dental stem cells was measured every 11 days after 1×10³ seeding. Sternness of various human dental stem cells was identified by using a clonogenic assay.

FIG. 6 is a diagram illustrating STRO-1 expression of human dental stem cells. DPSCs, PDLSCs, PAFSCs, MBMSCs, ABMSCs and MXBMSCs expressed mesenchymal stem cell marker-STRO-1. STRO-1 positive cells were appeared more frequently in the earlier passages than the later passages.

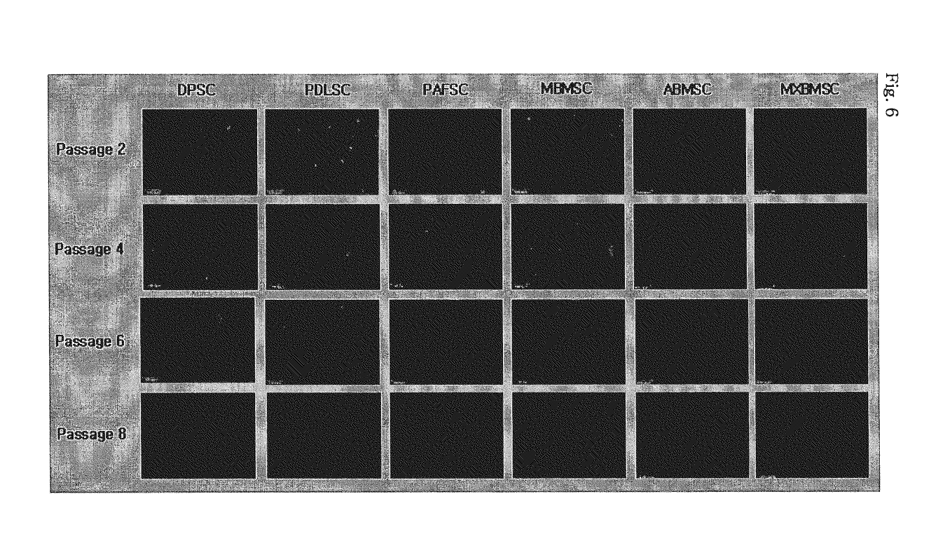

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Cell Culture

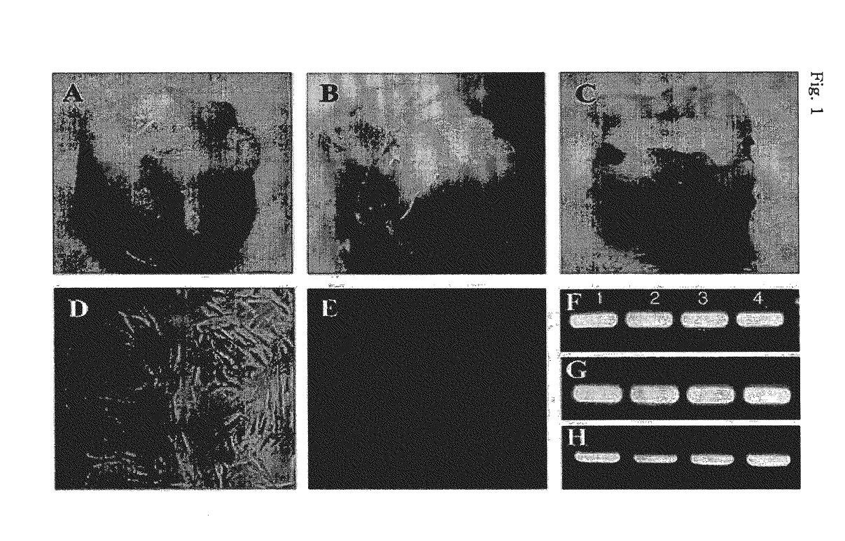

The present inventors collected normal and nondecayed human third molars that were impacted or extracted for orthodontic treatment purposes from 13 adults (18-35 years of age) under sufficient informed consent at the Seoul national University Dental Hospital, Seoul, South Korea. Bone marrow cells were released from the mandibular bone of the human volunteers (6-40 years of age). Dental pulp, periodontal ligament, periapical follicle and mandibular bone marrow tissues were obtained from teeth and circumference. The experimental protocol was approved by the Institutional Review Board (IRB) of the hospital. PDL was gently separated from the surface of the root and the dental pulp tissue was separated from the pulp chamber, which was revealed by cutting around the cemento-enamel junction with sterilized dental tissue burs (FIG. 1). Tooth germs at the root-forming stage were obtained and named periapical follicles. These were removed with a scalpel from where they attached to the root dentin. Osteotomized mandibular bone that had been disposed of following orthognathic surgery was utilized for analysis after obtaining written permission from the patients (6-40 years of age, four samples). The bone segments were digested in a solution of 3 mg/mL collagenase type I and 4 mg/mL dispase for 1 hour at 37° C. Single-cell suspensions were obtained by passing the cells through a 70-um cell strainer (Falcon, BD Labware, Franklin Lakes, N.J.). To isolate putative stem cells, single-cell suspensions (1×10⁴ cells) were seeded into 100 mm dishes (NUNC, Denmark) with alpha modification of Eagle's medium (alpha-MEM, GIBCO BRL, Carlsbad, Calif.) supplemented with 10% fetal calf serum (FCS: Gibco BRL), 100 uM/L ascorbic acid 2 phosphate (Sigma, St. Louis, Mo.), 2 mM/L glutamine, 100 U/mL penicillin and 100 ug/mL streptomycin (Gibco BRL) and then incubated at 37° C. in 5% carbon dioxide.

To assess colony-forming efficiency, day 10 cultures were fixed with 70% ethanol and then stained with 0.1% crystal violet. Aggregates of 50 or more cells were scored as colonies. After 4, 7 and 10 days of culture, the proliferation rates of stem cells were assessed by MTT assay.

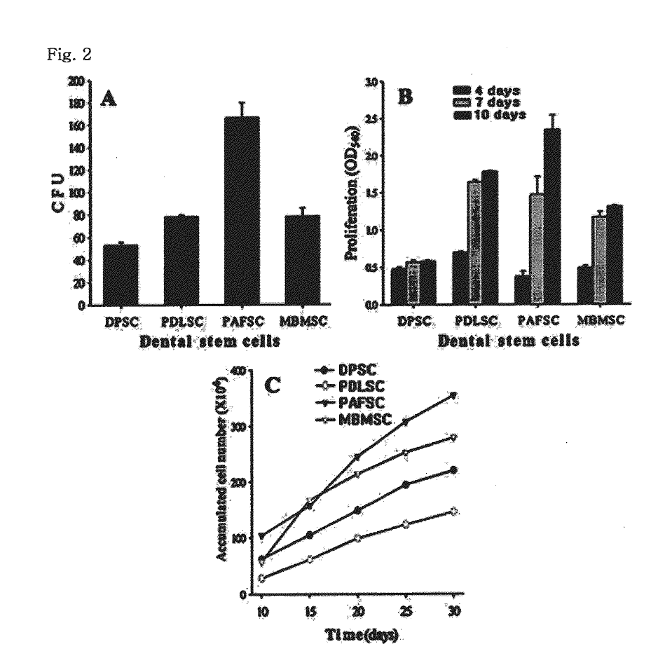

As a result, the isolated cells formed single-cell-derived colonies and most of the cells retained their fibroblastic spindle shape (FIG. 10). The stem cells were capable of forming adherent colonies, characteristic of other stromal stem cell populations (FIG. 2A). Compared to other dental stem cells, PAFSC formed twice the number of colonies for the same number of initial loading cells (FIG. 2A). To evaluate proliferation ability, passage 3 PDLSC, DPSC, PAFSC, and MBMSC were plated at 1000 cells/well and cultured for 4, 7, and 10 days with optimal culture medium. PAFSC shoed a higher proliferation rate (FIG. 2B) at day 10 and higher number of population doublings (FIG. 2C) compared to the others.

### Example 2

### Immunohistochemistry

Isolated putative stem cells from the PDL, dental pulp, periapical follicle, and mandibular bone (PDLSC, DPSC, PAFSC and MBMSC) were seeded on two-chamber slides (2×10⁴ cells/well: NUNC, Denmark) and cultured for 24 hours. After being washed in phosphate-buffered saline and fixed in 2% p-formaldehyde for 15 minutes, the samples were incubated with STRO-1 antibody (1:200, R&D System Inc., Minneapolis, Minn., USA) for 3 hours. The cells were subsequently incubated with goat secondary antibodies of anti-mouse IgG-Cy3 (Zymed laboratories, Carlsbad, Calif.) for one hour. Then the nuclei were stained in DAPI (2 ug/ml) for 30 minutes.

As a result, the isolated cell populations expressed the cell surface molecule STGRO-1, an MSC marker, previously found to be present in DPSC (FIG. 1E).

### Example 3

### Adipogenic Differentiation

For adipogenic differentiation, 3×10⁴ cells were plated in a 60 mm dish and cultured in optimal culture medium for 3 days. The medium was replaced with adipogenic medium, which was the optimal culture medium supplemented with 0.5 mM methylisobutylzantine, 0.5 uM hydrocortisone, and 60 uM indomethacin. The cells were cultured for additional 21 days. The adipogenic cultures were fixed in 70% ethanol for 15 minutes and stained with fresh Oil red O solution (Sigma) for 2 hours.

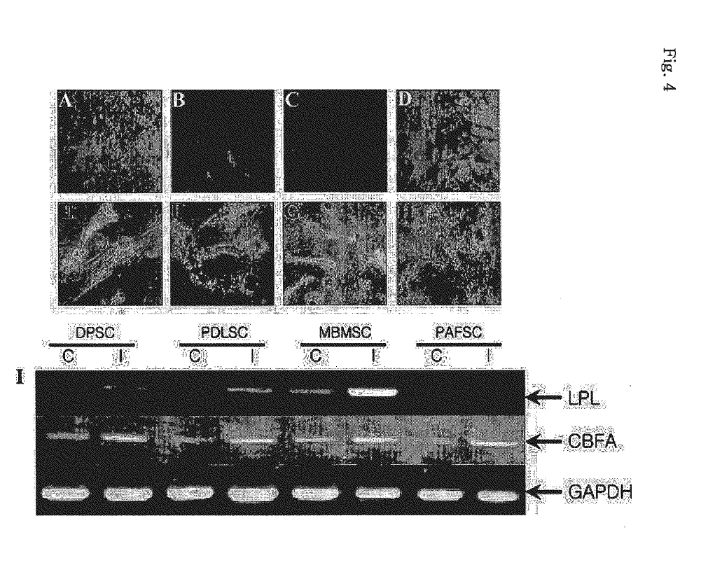

As a result, after 3 weeks in the induction medium, lipid accumulation was noted by Oil red O staining (FIG. 4E-H).

### Example 4

### Calcification

For calcification, 1×10⁴ cells were plated in a 60-mm dish and cultured in optimal culture medium for 3 days. They were then incubated with optimal culture medium supplemented with 5 mM β-glycerophosphate and 10 nM dexamethasone (Sigma) for 3 weeks to induce mineral formation. Mineral nodule formation was observed by staining the cultures with 40 mM Alizarin red (pH 4.2). the present inventors also quantified the amount of Alizarin red that bound to the mineral in each dish by destaining the samples in 10 mM sodium phosphate containing 10% cetylpyridinium chloride (pH 7) for 15 minutes at room temperature. The amount of Alizarin red in the destaining solution was measured at 562 nm and compared with a standard solution of the dye.

As a result, three-seek-old cultures of dental stem cells grown in the presence of β-glycerophosphate and dexamethasone demonstrated the capacity to form Alizarin red positive condensed nodules with a high level of calcium. The deposits from DPSCs and PAFSCs were sparsely scattered over the adherent layer, whereas MBMSCs and PDLSCs cultures produced extensive sheets of calcified deposits throughout the adherent layer after 2 weeks of induction (FIG. 4A-D). Quantification of the amount of Alizarin red after 21 days of induction showed that the accumulated calcium levels in cultures of MBMSC and PDLSC were higher than in other cultures (Table 1).

TABLE 1
Mineralization ability of human dental stem cells

| Dental stem cells | DPSC | PDLSC | PAFSC | MBMSC |
|---|---|---|---|---|
| Alizarin red (mM) | 0.23 | 54.62 | 3.08 | 61.71 |
| ALP activity | 981 | 1300 | 875 | 1536 |
| (uM p-NP/mg | | | | |
| protein/min) | | | | |

* Statistical significance was accepted at p < 0.05

After 21 days of induction, Alizarin red staining in DPSCs was similar to that of PAFSCs at 14 days of induction but mineralization was not induced (FIG. 1A). The above results indicate that human dental stem cells have different calcium accumulation capacity and in particular calcium accumulation of DPSCs is lower than those of others.

### Example 5

### Alkaline Phosphatase (ALP) Activity

The present inventors assayed enzyme activity of ALP, which is widely used as a marker of biomineralization. ALP cleaves non-specifically phosphate ions from compounds and its activity is increased at the mineralization surface when a mineral crystal is growing.

Quantitative ALP activity was determined by an assay based on the hydrolysis of p-nitrophenylphosphate (p-NPP) to p-nitrophenol (p-NP). Dental stem cells cultured in the induction media for 21 days were harvested in lysis buffer solution containing 0.1% Triton X-100. The lysate (60 ul) of each dental stem cell was added to 60 ul of 0.2 M diethanolamine and 30 mM p-NPP. The samples were then incubated at 37° C. for 30 minutes and the reaction was stopped by addition of 60 ul of 0.3 N sodium hydroxide. ALP activity was measured colorimetrically with p-nitrophenol as substrate with the use of ELISA (enzyme-linked immunosobent assay) reader at 410 nm. Enzyme activity is expressed as uM/mg protein/minute.

As a result, after 21 days of induction, ALP activity was high in MBMSCs and PDLSCs, which shoed a high level of calcium accumulation. ALP activity of DPSCs was higher than PAFSCs although accumulated calcium level of DPSCs was lower than PAFSCs (Table 1).

### Example 6

### RT-PCR

Total RNA was prepared using Trizol reagent (Invitrogen, USA) according to the manufacturer's protocol. First-strand cDNA was synthesized using a first-strand cDNA synthesis kit (Invitrogen, USA). The primer set for PCR included CD29 (sense, 5′-AATGAAGGGCGTGTTGGTAG-3′: antisense, 5′-CGTTGCTGGCTTCACAAGTA-3′), CD44 (sense, 5′-GCAATGCTTCTCAGACCACA-3′; antisense, 5′-CTGGCCAATGTAGTTCACAG-3′), LPL (lipoprotein lipase; sense, 5′-ATGGAGAGCAAAGCCCTGCTC-3′; antisense, 5′-GTTAGGTCCAGCTGGATCGAG-3′), CBFA1 (CCAAT-binding transcription factor subunit A; sense, 5′-CAGTTCCCAAGCATTTCATCC-3′; antisense, 5′-TCAATATGGTCGCCAAACAG-3′), GAPDH (glyceraldehydes-3-phosphate dehydrogenase; sense, 5′-AGCCGCATCTTCTTTGCGTC-3′; antisense, 5′-TCATATTTGGCAGGTTTTTCT-3′). The PCR reactions were preincubated in a PCR Mastercycler gradient (Eppendorf, Hamburg, Germany) at 95° C. for 3 minutes and then cycled 33 times at 95° C./30 sec, 55° C./45 sec, and 72° C./60 sec, followed by a final 10-minute extension at 72° C. The products were separated by electrophoresis on a 1% agarose gel and visualized by ultraviolet induced fluorescence.

As a result, the dental stem cells of the invention expressed the MSC markers CD29 and CD44 (FIG. 1F, G). The expressions of the adipocyte specific transcript, LPL, and the osteogenesis specific transcript, CBFA, were also increased in the induction group (FIG. 4I).

### Example 7

### Optimum Culture Conditions

Single-cell suspensions (1×10³ cells/well) of PDL, dental pulp, periapical follicle, and mandibular bone were seeded into a 96-well culture plate (NUNC, Roskilde, Denmark) with culture medium. To optimize the culture conditions, three types of media were used; Roswell Park memorial institute (RPMI) medium, Dulbecco's modified Eagle's medium (DMEM) and alpha-MEM supplemented with 2 mM/L glutamine, 100 U/mL penicillin and 100 ug/ml streptomycin (Gibco BRL). Fetal calf serum (FCS) was added at 10% or 20% concentrations and different concentrations of ascorbic acid were tested (0, 50, 100 and 200 uM). Culture medium was changed at 2- to 3-day intervals.

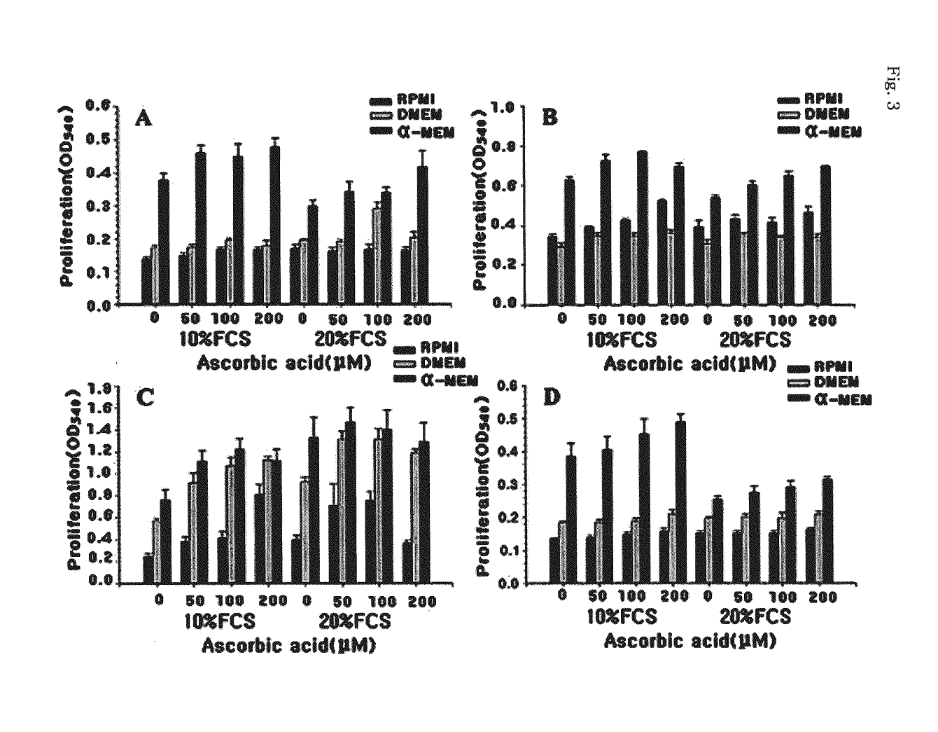

Almost all of the dental stem cells showed optimum growth when cultured in alpha-MEM supplemented with 10% FCS, 100 uM ascorbic acid, 2 mM/L glutamine, 100 U/mL penicillin and 100 ug/mL streptomycin. PAFSC, however, showed optimum growth when cultured in alpha-MEM supplemented with 20% FCS and 50 uM ascorbic acid (FIG. 3).

Each experiment was repeated at least three times. Results were expressed as the mean±standard deviation. All data were statistically analyzed using the one-way analysis of variance (ANOVA). Statistical significance was accepted at p<0.05.

### Example 8

### In Vitro Expandability of Various Dental Stem Cells

The present inventors isolated postnatal stem cells from various human dental tissues, such as dental pulp (DPSCs), periodontal ligament (PDLSCs), periapical follicle (PAFSCs), mandibular bone (MBMSCs), alveolar bone (ABMSCs) and maxillary bone (MXBMSCs). PAFSCs, ABMSCs, and MXBMSCs have not been previously reported. Then, the inventors evaluated cell proliferation at each passage. To identify the maintenance of stemness in the various dental stem cells, the present inventors performed a clonogenic assay and immunohistochemistry for the expression of STRO-1 molecule.

1. Cell Culture

Extracted human third molars were collected from adults (18-35 years of age) at the Seoul National University Dental Hospital under the guideline of experimental protocol approved by the IRB (Institutional Review Board) of Seoul National University Dental Hospital (Seoul, South Korea). PDL was separated from the surface of the root and pulp was isolated from the pulp chamber, which was revealed by cutting around the cemento-enamel junction with sterilized dental fissure burs. Periapical follicular cells were separated with a scalpel from where attached to the root dentin. Bone marrow cells were processed from the mandibular bone, alveolar bone, and maxillary bone of human volunteers (6-40 years of age). These dental tissues were cultured according to the method described by Gronthos et al. (S. Gronthos M. Mankani, J. Brahim, P. G. Robey and S. Shi: PNAS 91(25) (2000), P. 13625).

2. Subculture and Clonogenic Assay

The total cell number of the cultured cells was counted and replated at 3×10⁴ cells every 5 days until their expansion potential was lost. To assess colony-forming efficiency, dental stem cells of each passage were seeded at 1×10³ into a 100 mm culture dish. Day 11 cultures were fixed with 70% ethanol and then stained with 0.1% crystal violet by the method described by Gronthos et al (S. Gronthos, M. Mankani, J. Brahim, P. G, Robey and S. Shi; PNAS 91(25) (2000), P 13625). Colony forming efficiency was expressed as colony forming units (CFUs), which indicates colonies per 1×10³ cells plated. Each experiment was repeated at least three times. Results were expressed as the mean±deviation. All data were statistically analyzed using the one-way analysis of variance (ANOVA). Statistical significance was accepted at p<0.05.

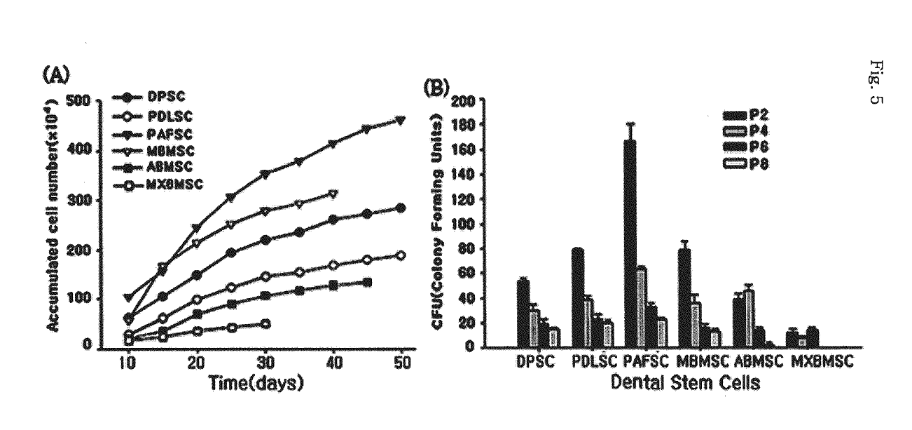

As a result, the proliferative ability of DPSCs, PDLSCs and PAFSCs was retained even at passage 10. PAFSCs had the greatest proliferation capacity and MXBMSCs was the lowest one (FIG. 5A). All postnatal dental stem cells could generate colony at each passage until passage 10 (FIG. 5B). The colony was declined as passage increased. The CFUs of ABMSCs and MXBMSCs were lower than other stem cells. In particular, PAFSCs obtained from periapical follicle were superior in their colony forming potential among the tested dental stem cells in this invention.

3. Immunohistochemistry

To determine cells expressing specific marker proteins, putative stem cells obtained from dental tissues were seeded at 2×10⁴ cells/well on two-chamber slides and cultured for 24 hours. After being fixed in 2% paraformaldehyde, the samples were then incubated with STRO-1 antibody (1:200, R&D System Inc., USA). The cells were subsequently incubated with goat secondary antibodies of anti-mouse IgG-Cy3 (Zymed laboratories, USA). Then, the nuclei were stained with DAPI (2 ug/ml).

STRO-1 positive cells were located in the tissue of pulp, PDL, periapical follicle, mandibular bone, alveolar bone, and maxillary bone by immunohistochemical staining (FIG. 5). STRO-1 positive cells were much more frequently detected in DPSCs, PDLSCs, PAFSCs and MBMSCs to the passage 8 than AMBSCs and MABMSCs.

Comparing proliferative capacity, DPSCs, PDLSCs, PFSC, MBMSCs and ABMSCs retained their ability to proliferate even at passage 10 except MXBMSCs. It seems that the stem cells from bone have difficult maintaining their stemness for an extended period of time than those from the other dental tissues. During repeated passaging, each dental stem cell type is different in their colony forming efficiency and STRO-1 expression according to the dental tissue source. In particular, PAFSCs are excellent with regard to proliferation, colony forming efficiency and STRO-1 expression. These results provide important information for the selection of the optimal dental tissue as a source of cells for improved clinical usage.

### INDUSTRIAL APPLICABILITY

As explained hereinbefore, the method for neuronal regeneration using bone marrow originated stem cell transplantation and the screening method of a neuronal regeneration material by using ex vivo model of the spinal cord section can be effectively used for the development of a therapeutic agent for neuronal diseases.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. Postnatal dental stem cells having a mineralization ability, which are isolated from dental pulp (DP), periodontal ligament (PDL), periapical follicle (PAF) or mandibular bone marrow (MBM).

2. The postnatal dental stem cells according to claim 1, wherein the MBM originated stem cells (MBMSCs) and PDL originated stem cells (PDLSCs) exhibit higher calcium accumulation and ALP activity than DP originated stem cells (DPSCs) and PAF originated stem cells (PAFSCs).

3. The postnatal dental stem cells according to claim 1, wherein the stem cells express Cbfa 1, a known transcription factor associated with differentiation of osteoblasts.

4. The postnatal dental stem cells according to claim 1, wherein the stem cells express LPL, the adipocyte-specific transcript.

5. The postnatal dental stem cells according to claim 1, wherein the stem cells express STRO-1, CD29 or CD44, the mesenchymal stem cell markers.

6. The postnatal dental stem cells according to claim 1, wherein the stem cells could generate colony even at passage 10.

7. The postnatal dental stem cells according to claim 1, wherein the PAFSCs form twice the number of colonies for the same number of initial loading cells compared to other dental stem cells, DPSCs, PDLSCs or MBMSCs.

8. The postnatal dental stem cells according to claim 1, wherein the stem cells are used for regeneration of injured teeth.

9. The postnatal dental stem cells according to claim 1, wherein the MBMSCs and PDLSCs exhibit higher mineralization ability than DPSCs and PDLSCs in mineralization induction medium.

10. The postnatal dental stem cells according to claim 9, wherein the mineralization induction medium is prepared by adding 1-10 uM β-glycerophosphate and 1-50 nM dexamethasone to the dental stem cell culture medium.

11. The postnatal dental stem cells according to claim 9, wherein the mineralization is evaluated by the index selected from the group consisting of calcium accumulation, ALP activity and Cbfa expression.

12. The postnatal dental stem cells according to claim 1, wherein the stem cells are differentiated into osteoblasts and adipocytes by culture.

13. A method for isolating the postnatal dental stem cells from dental pulp (DPSCs), periodontal ligament (PDLSCs), periapical follicle (PAFSCs) or mandibular bone marrow (MBMSCs), comprising the following steps:
1) separating dental pulp (DPSCs), periodontal ligament (PDLSCs), periapical follicle (PAFSCs) or mandibular bone marrow (MBMSCs) from adult teeth;
2) digesting the tissues obtained in step 1); and
3) filtering the digested tissues of step 2).

14. The method for isolating the postnatal dental stem cells according to claim 13, wherein the periapical follicle is tooth germ at the root forming stage.

15. The method for isolating the postnatal dental stem cells according to claim 13, wherein the tissue was digested in a solution of 1-10 mg/ml collagenase type 1 and 1-10 mg/ml dispase at 37° C. for up to 2 hours.

16. The method for isolating the postnatal stem cells according to claim 13, wherein the cells were passed through a cell strainer to obtain single cell suspensions.

17. A method for culturing the postnatal dental stem cells isolated from dental pulp (DP), periodontal ligament (PDL) and mandibular bone marrow (MBM) in alpha-MEM supplemented with 10% FCS, 50-200 uM ascorbic acid, 1-10 mM/L glutamine, 50-200 U/mL penicillin and 50-200 ug/mL streptomycin.

18. A method for culturing the postnatal dental stem cells isolated from periapical follicle (PAF) in alpha-MEM supplemented with 20% FCS, 10-200 uM ascorbic acid, 1-10 mM/L glutamine, 50-200 U/mL penicillin and 50-200 ug/mL streptomycin.

19. A method for inducing mineralization of the postnatal stem cells, including the step of adding 1-10 mM β-glycerophosphate and 1-50 nM dexamethasone to the postnatal dental stem cells of claim 1.
